(19) United States
(12) Patent Application Publication
(10) Pub. No.: US 2010/0094237 A1
(43) Pub. Date: Apr. 15, 2010

(54) EASY ON-OFF DEVICE TO SUPPORT AND CHANGE ABSORPTION PADS FOR PATIENTS SUFFERING FROM INCONTINENCE

(75) Inventors: GEOFFREY MARGOLIS, Los Angeles, CA (US); Mark Kelly, Santa Monica, CA (US); Peter Loisides, Santa Monica, CA (US)

Correspondence Address: PILLSBURY WINTHROP SHAW PITTMAN LLP, P.O BOX 10500, McLean, VA, 22102, US

(73) Assignee: UROLOGY INVENTIONS, L.L.C., Los Angeles, CA (US)

(21) Appl. No.: 12/572,262

(22) Filed: Oct. 1, 2009

Related U.S. Application Data
(60) Provisional application No. 61/102,342, filed on Oct. 2, 2008.
(60) Provisional application No. 61/218,372, filed on Jun. 18, 2009.

(51) Int. Cl. A61F 13/49

(52) U.S. Cl. 604/385.01

(57) ABSTRACT

Embodiments of the present invention are directed to apparati and associated methods for an easy on-off device to support and change absorption pads for patients suffering from incontinence.

### BACKGROUND OF THE INVENTION

1. Field of the Invention

Millions suffer from varying degrees of incontinence and a fair percentage of elderly incontinent patients are bed ridden. In order to be able to function reasonably normally these incontinent patients use various types of absorbent pads that continuously absorb the urine emanating from their bladder.

Generally commercially available urine absorbent pads are available in four forms: as insertable absorbent pads to be placed between the body and regular underwear; as absorbent pads that are integrated into disposable underwear; as absorbent pads that are integrated into disposable diaper like garments, requiring the front and rear parts of the diaper to be fastened along the user's sides so as to maintain the diaper in close contact with the user; and as absorbent pads that are integrated into disposable diaper like garments, requiring the use of a belt attached to the diaper to support and maintain the diaper in close contact with the user.

Whereas these commercially available products are extremely helpful to people suffering from incontinence, they nevertheless have various shortcomings. Thus, in all cases, changing of the urine laden absorption pad requires first dropping or removing the patient's underwear. This is particularly difficult if the patient is bed-ridden and supine. Similarly, many of the above product forms cannot accommodate various sized patients, and, of course, the insertable pad form is limited in its urine uptake capacity. Therefore, there is a need for an improved easy on-off device to support and change absorption pads for patients suffering from incontinence.

2. Description of Related Art

There are many patents directed at the design of improved garments for use by incontinent persons, see, for example U.S. Pat. No. 3,162,196 (Garment for incontinent individuals); U.S. Pat. No. 4,338,939 (Incontinence pants); U.S. Pat. No. 5,074,853 (Male incontinence diaper); U.S. Pat. No. 5,098,419 (Undergarment to be worn by incontinent person); D352,352 (Incontinence brief), and US Patent Application US 2008/0086105 (Protective underwear with abdominal support).

Certain patents are directed improved methods of fastening the absorbent article around the patient, such as U.S. Pat. No. 5,984,911 (Absorbent article having an improved fastening system); US Patent Application US 2007/0299418 (Fastening means in the form of a belt for an absorbent article) and US Patent Application US 2008/0097369 (Absorbent article comprising a belt). None of these patents are directed at improving the issues described above.

### SUMMARY OF THE INVENTION

The novel device to support and allow for the changing of absorption pads disclosed in this invention provides unique benefits and overcomes deficiencies in current products.

1. The device has an easy on-off feature so that a urine laden absorbable pad can be easily changed without the necessity of removing underwear.

2. The urine pad or even the complete device can be easily changed on a supine person.

3. The device can be adjusted to accommodate different patient sizes, and the tension can be adjusted on the pad carrying support section.

The device either holds a disposable urine absorbable pad or has a urine absorbable pad integrated into the structure of the device. The device is an absorption pad apparatus for use on a person's pelvis. The absorption pad apparatus is for use on either male or female users. The absorption pad apparatus comprises a triangular shaped pelvic unit, a cloth extension from the apex of the pelvic unit, onto which is attached an elastic extension and two elastic straps, a first strap and a second strap, a first strap connector and a second strap connector, and at least one disposable absorption pad that is replaceable and removable.

The pelvic unit fits around the front of a person's pelvis, with the cloth extension portion of the pelvic unit being directed in between the legs of the person within close proximity to the person's body. Depending upon the length of the cloth extension, the first and second straps can either be wrapped around the patient's waist and secured to the wide elastic band at the top of the pelvic unit; or wrapped across the person's buttocks and over the hip area and then secured to the wide elastic band at the top of the pelvic unit. When both the first and the second straps are connected to the first and second strap connectors (using for example, Velcro® material), the absorption pad apparatus is secured to the person.

The absorption pad apparatus further includes an absorption pad that can either be disposable or integral to the pelvic unit. The absorption pad is of dimensions that cover the area which is generally likely to become in contact with the urine from the person wearing the absorption pad apparatus. The dimensions of the absorption pad are such that it will effectively absorb urine regardless of the gender of the person wearing the absorption pad apparatus.

The entire absorption pad apparatus can be easily put onto a supine patient by passing the cloth extension between the patient's legs, and simply sliding the elastic straps up and under the patient's buttock. Thus, it is not necessary to lift the patient's torso to place the apparatus on the patient—as is required by the currently available products. The two straps are then passed around the patient's waist or across the patient's buttocks and over the patient's hips and are connected to the first and second strap connectors. Reversing this sequence permits the apparatus to be easily removed from the patient.

By adjusting the horizontal location of the connection contact between the straps and strap connectors, the device can accommodate different sized patients. By adjusting the vertical location of the connection contact between the straps and strap connectors, the device can adjust the contact tension between the pelvic unit and the patient.

The release of at least one strap allows a disposable absorption pad to be removed without the necessity of removing the absorption pad apparatus from the patient or even completely removing the patient's underwear.

### BRIEF DESCRIPTION OF THE DRAWINGS

FIG. 1 is a laid-open view of the absorption pad apparatus.

FIG. 2a is a front view of the absorption pad apparatus as applied to a person.

FIG. 2b is a rear view of the absorption pad apparatus as applied to a person.

FIG. 3 is a laid open view of another embodiment of the absorption pad apparatus.

### DETAILED DESCRIPTION OF A PREFERRED EMBODIMENT OF THE INVENTION

Embodiments of the present invention are directed to apparati (and associated methods) for an easy on-off device to support and change absorption pads for patients suffering from incontinence.

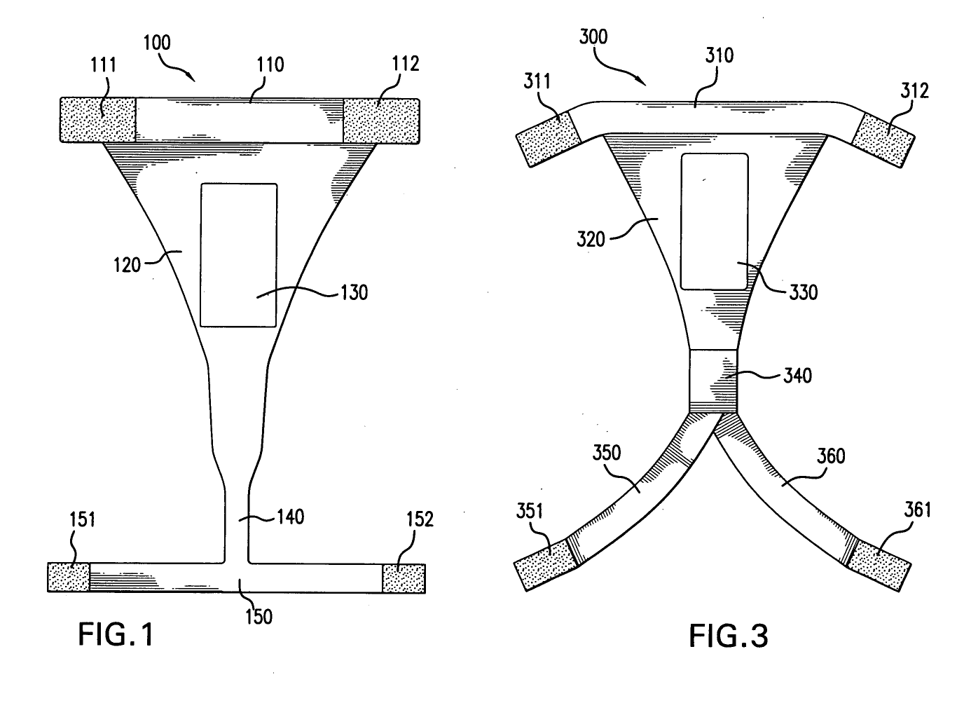

FIG. 1 shows the preferred embodiment of an absorption pad apparatus 100. This embodiment of the absorption pad apparatus 100 for use on the pelvic area of a human comprises a generally elongated support unit 120, which is the main support structure. The support unit 120 is triangular in shape, with a wider dimension on one side, and a narrower dimension at the other. In this embodiment, the support unit 120 is made from a soft, flexible, elastic-type cloth, although other types of materials that are commonly used for clothing are contemplated as well. At the wider dimension at the top of the support unit 120 there is a first strap 110, which is similar to a waist-band and may be adjustable in size to accommodate differently sized individuals that wear the absorption pad apparatus 100. At each end of the first strap 110 are attached two connectors 111 and 112, with the first connector 111 and the second connector 112 comprising a Velcro® connecting strip; however, other connecting techniques and materials are also contemplated such as snaps, buttons, and adhesives, as well as other connecting products that are known in the art. At the narrower dimension at the bottom of the support unit 120 there is an extended portion 140. The extended portion 140 is designed to extend between the wearer's legs and up the back of the person wearing the absorption pad 100. A second strap 150 connects to the extended portion 140 adjacent a center point of the second strap 150. The second strap 150 is similar to a waist-band and may be adjustable in size to accommodate differently sized individuals that wear the absorption pad apparatus 100. Adjacent each end of the second strap 150 are attached two connectors 151 and 152, with the first connector 151 (the third connector of the absorption pad apparatus) of the second strap 150 and the second connector 152 (the fourth connector of the absorption pad apparatus) of the second strap 150 comprising a Velcro® connecting strip; however, other connecting techniques and materials are also contemplated such as snaps, buttons, and adhesives, as well as other connecting products that are known in the art. When the absorption pad apparatus 100 is worn by an individual, the first connector 151 of the second strap 150 releasably connects to the first connector 111 on first strap 110, and the second connector 152 of the second strap 150 releasably connects to the second connector 112 on the first strap 110.

It is also contemplated that the extended portion 140 could be an extension strap having a first extension strap-end and a second extension strap-end, wherein the extension strap 140 is connected at the first extension strap-end of the extension strap 140 to the bottom of the support unit 120, and the extension strap 140 extends for a distance that places the second extension strap-end near the waist of a person when that person is wearing the absorption pad apparatus 100. In some cases, the extension strap 140 may be adjustable to better accommodate differently sized individuals that wear the absorption pad apparatus 100. A second strap 150 connects to the second extension strap-end of the extension strap 140 near a center point of the second strap 150. The second strap 150 is similar to a waist-band and may be adjustable in size to accommodate differently sized individuals that wear the absorption pad apparatus 100. At each end of the second strap 150 are attached two connectors 151 and 152, with the first connector 151 of the second strap 150 and the second connector 152 of the second strap 150 comprising a Velcro® connecting strip; however, other connecting techniques and materials are also contemplated such as snaps, buttons, and adhesives, as well as other connecting products that are known in the art. When the absorption pad apparatus 100 is worn by an individual, the first connector 151 of the second strap 150 releasably connects to the first connector 111 on first strap 110, and the second connector 152 of the second strap 150 releasably connects to the second connector 112 on the first strap 110.

As shown in FIG. 1, the support unit 120 carries at least one absorption pad 130. The absorption pad 130 is removable and attachable at any area on or inside the support unit 120. In this manner, the absorption pad 130 can be placed more accurately to provide more efficient absorption of urine or other bodily fluids. Generally, the absorption pad 130 is centrally located on the support unit 120.

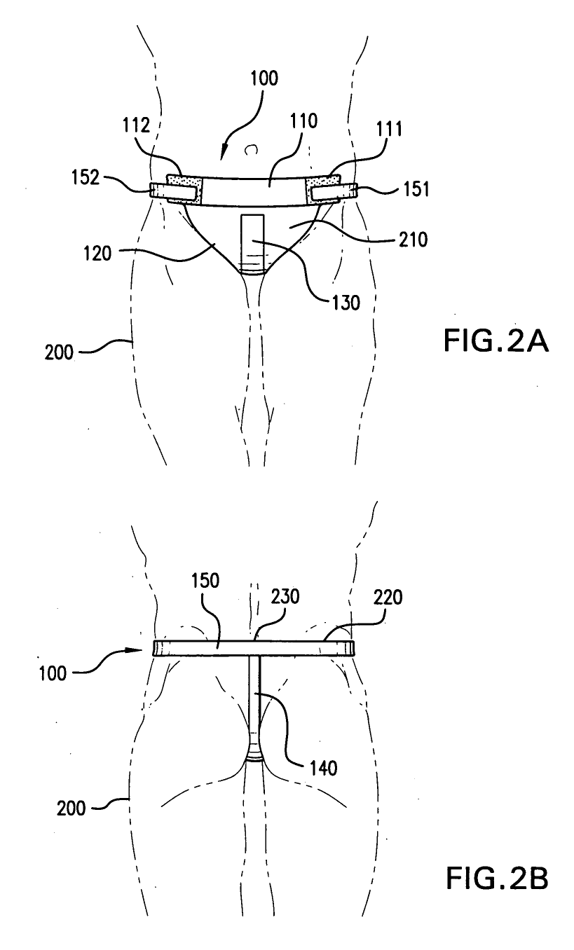

Referring to FIG. 2A, the absorption pad apparatus 100 is secured to the wearer 200 by connecting the first connector 111 on first strap 110 to the first connector 151 of the second strap 150 and by connecting the second connector 112 on the first strap 110 to the second connector 152 of the second strap 150. Once the connections of the first connector 111 on first strap 110 to the first connector 151 of the second strap 150 and the second connector 112 on the first strap 110 to the second connector 152 of the second strap 150 are made, the support unit 120 engages the genitalia 210 of the wearer 200. The support unit 120 houses the absorption pad 130, which is located in the support unit 120 to contact the genitalia 210 of the wearer 200. The absorption pad 130 is removable from the support unit 120 and is capable of being placed at any point within the support unit 120. The selectable placement of the absorption pad 130 allows for the absorption pad 130 to be placed against various areas of the genitalia 210 of the wearer 200. The selectable placement of the absorption pad 130 can be based upon the wearer's 200 particular anatomy and bodily fluids absorption needs. This is an important feature of the absorption pad apparatus 100 because the wearer 200 may be a male or a female, which require different absorption pad 130 placement to accommodate proper absorption of fluids in the area.

FIG. 2B illustrates the back view of a wearer 200 of the absorption pad apparatus 100, as used on the pelvic area of a human wearer 200. The extension strap 140 is connected at the first extension strap-end of the extension strap 140 to the bottom of the support unit 120 (see FIG. 2a), and the extension strap 140 extends for a distance that places the second extension strap-end near the waist 230 of a wearer 200 when that person is wearing the absorption pad apparatus 100. In some cases, the extension strap 140 may be adjustable to better accommodate differently sized individuals that wear the absorption pad apparatus 100. A second strap 150 connects to the second extension strap-end of the extension strap 140 near a center point of the second strap 150. The second strap 150 is similar to a waist-band and may be adjustable in size to accommodate differently sized individuals that wear the absorption pad apparatus 100. The second strap 150 extends across the lower back 220 of the wearer 200.

The method for applying the absorption pad apparatus 100 to a patient (wearer 200) is as follows:

The support unit 120 of the absorption pad apparatus 100 described in FIGS. 1-2b with absorption pad 130 is placed and held directly over the genitalia 210 of the wearer 200 with a first hand of the individual applying the absorption pad apparatus 100, while passing the extension strap 140 and the second strap 150 between the wearer's legs towards the wearer's posterior waist region 230, and then wrapping the second strap 150 around the wearer's lower back 220 and attaching the first connector 151 of the second strap 150 to the first connector 111 on first strap 110, and the second connector 152 of the second strap 150 to the second connector 112 on the first strap 110 using the individual's other hand. In this simple manner, the device can be easily set in place by either the patient, nurse, or other individual because the absorption pad apparatus 100 has easy on/off characteristics.

A method for replacing the absorption pad 130 contained in the absorption pad apparatus 100 to a supine patient (wearer 200) is as follows:

The individual replacing the absorption pad 130 disconnects either the first connectors 111, 151 or the second connectors 112, 152, or all the connectors 111, 151, 112, 152 from each other. The individual replacing the absorption pad 130 then removes the absorption pad 130 from the support unit 120. The individual replacing the absorption pad 130 then places a new absorption pad 130 into the support unit 120. The individual replacing the absorption pad 130 then reconnects any of the connectors 111, 151, 112, 152 that the individual previously disconnected.

FIG. 3 is an alternative embodiment of the absorption pad apparatus 300 for use on the pelvic area of a human comprises a generally elongated support unit 320, which is the main support structure. The support unit 320 is triangular in shape, with a wider dimension on one side, and a narrower dimension at the other. In this embodiment, the support unit 320 is made from a soft, flexible, elastic-type cloth, although other types of materials that are commonly used for clothing are contemplated as well. At the wider dimension at the top of the support unit 320 there is a first strap 310, which is similar to a waist-band and may be adjustable in size to accommodate differently sized individuals that wear the absorption pad apparatus 300. Adjacent each end of the first strap 310 are attached two connectors 311 and 312, with the first connector 311 and the second connector 312 comprising a Velcro® connecting strip; however, other connecting techniques and materials are also contemplated such as snaps, buttons, and adhesives, as well as other connecting products that are known in the art. At the narrower dimension at the bottom of the support unit 320 there is an extended portion 340. The extended portion 340 extends for a distance that places the end of the extended portion 340 adjacent to the sacroiliac region of a person when that person is wearing the absorption pad apparatus 300. A second strap 350 and a third strap 360, which both straps 350 and 360 have a proximal end and a distal end, respectively, extend from the extended portion 340. More specifically, the proximal end of the second strap 350 is connected to the extended portion 340, and the proximal end of the third strap 360 is connected to the extended portion 340. The proximal end of the second strap 350 may overlap the proximal end of the third strap 360 at the connection to the extended portion 340, or the proximal end of the second strap 350 may reside next to the proximal end of the third strap 360 at the connection to the extended portion 340. Adjacent the distal end of the second strap 350 is a second strap connector 351, and adjacent the distal end of the third strap 360 is a third strap connector 361. In this embodiment, the second strap connector 351 and the third strap connector 361 each comprise a Velcro® connecting strip; however, other connecting techniques and materials are also contemplated such as snaps, buttons, and adhesives, as well as other connecting products that are known in the art. When the absorption pad apparatus 300 is worn by an individual, the second strap connector 351 releasably connects to the first connector 311 on first strap 310, and the third strap connector 361 releasably connects to the second connector 312 on the first strap 310. In some cases, the second strap 350 and the third strap 360 may be adjustable to better accommodate differently sized individuals that wear the absorption pad apparatus 300.

It is also contemplated that the extended portion could be an extension strap having a first extension strap-end and a second extension strap-end, wherein the extension strap 340 is connected at the first extension strap-end of the extension strap 340 to the bottom of the support unit 320, and the extension strap 340 extends for a distance that places the second extension strap-end near the sacroiliac region of a person when that person is wearing the absorption pad apparatus 300. In some cases, the extension strap 340 may be adjustable to better accommodate differently sized individuals that wear the absorption pad apparatus 300. The second strap 350 and a third strap 160 extend from the extension strap 340, and connect to the first and second connectors 311 and 312 in a similar way described above.

As shown in FIG. 3, the support unit 320 carries at least one absorption pad 330. The absorption paid 330 is removable and attachable at any area on or inside the support unit 320. In this manner, the absorption paid 330 can be paced more accurately to provide more efficient absorption of urine or other bodily fluids. Generally the absorption pad 330 is centrally located on the support unit 320.

It is understood that one or more of the components described herein in connection with a specific embodiment may be used in conjunction with one or more of the components described in connection with a different specific embodiment. Furthermore, other bodily fluids besides urine is contemplated as being absorbed by the absorption apparatus embodiments as disclosed herein. Those other bodily fluids, for example, may comprise blood, sebaceous fluid, or other fluids resulting from the human body.

While the description above refers to particular embodiments of the present invention, it will be understood that many modifications may be made without departing from the spirit and scope thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. An easy on, easy off absorption pad apparatus for use on a human pelvic area of a wearer, the absorption pad apparatus comprising:
a first generally elongated strap having a first connector adjacent an end thereof and a second connector adjacent another end thereof;
a generally elongated support unit extending from the first strap;
at least one absorption pad removably joined to said generally elongated support unit; and
a second generally elongated strap attached to said support unit generally opposite to said first strap, and having a third connector adjacent an end thereof and a fourth connector adjacent another end thereof;
wherein the first and second connectors are releasably connected to the third and fourth connectors so as to generally encircle the pelvic area of the wearer and to apply a controllable amount of pressure of the absorption pad to the wearer's pelvic area.

2. The absorption pad apparatus of claim 1 wherein the at least one absorption pad is disposed in a generally central location.

3. The absorption pad apparatus of claim 2 the generally elongated support unit has a generally triangular configuration.

4. The absorption pad apparatus of claim 1 wherein the at least one absorption pad is placed adjacent the wearer's genitalia.

5. The absorption pad apparatus of claim 1 wherein the support unit has a generally triangular shape with an extending portion opposite the first strap.

6. A easy on, easy off absorption pad apparatus for use on a human pelvic area of a wearer, the absorption pad apparatus comprising:
a first strap having a set of connectors comprising a first connector and a second connector, the first connector being carried on a first end of the first strap, and the second connector being carried on a second end of the first strap;
a generally elongated support unit extending from the first strap;
at least one absorption pad removably joined to said generally elongated support unit;
a second strap having a proximal end and a distal end, the second strap extending from the support unit, and having a second strap connector adjacent the distal end of the second strap;
the second strap connector being releasably connectable to the first connector of the first strap;
a third strap having a proximal end and a distal end, the third strap extending from the support unit, and having a third strap connector adjacent the distal end of the third strap; and
the third strap connector being releasably connectable to the second connector of the first strap,
wherein, when said second and third straps are connected to said first strap, said support unit is positioned adjacent the wearer's pelvic area.

7. The absorption pad apparatus of claim 6 wherein the at least one absorption pad is disposed in a generally central location.

8. The absorption pad apparatus of claim 7 the generally elongated support unit has a generally triangular configuration.

9. The absorption pad apparatus of claim 6 wherein the at least one absorption pad is placed adjacent the wearer's genitalia.

10. The absorption pad apparatus of claim 6 wherein the support unit has a generally triangular shape with an extending portion opposite the first strap.

11. A method for easy application and provision of an absorption pad apparatus to the pelvic area of a person, said method comprising the steps of
(a) placing a support unit over a person's anterior pelvic region;
(b) passing an extension strap, attached to said support unit between the person's legs, such that said extension strap generally reaches the lower back region of the person;
(c) wrapping a first adjustable strap, attached to said extension strap, over the person's hip region and securing said first adjustable strap to the anterior support unit;
(d) wrapping a second adjustable strap, also attached to said extension strap, over the person's other hip region and securing said second adjustable strap to the anterior support unit;
(e) said support unit and said extension strap having one or more absorption pads located adjacent the pelvic area, said absorption pads being selectively positionable on said support unit; and
(f) adjusting said first and second adjustable straps to secure and supply pressure to both the support unit and extension strap against the person's pelvic area.

12. A method, according to claim 11, wherein said extension strap generally reaches the sacroiliac joint of the person.

13. A method, according to claim 11, wherein said extension strap is of sufficient length so as to generally reach the waist of the person.
